(19) United States
(12) Patent Application Publication
(10) Pub. No.: US 2013/0202668 A1
(43) Pub. Date: Aug. 8, 2013

(54) Personal Towelette with Cooling Composition for Relief of Heat Symptoms

(71) Applicants: Deborah Bolner Prost, San Antonio, TX (US); Linda K. Caldwell, San Antonio, TX (US)

(72) Inventors: Deborah Bolner Prost, San Antonio, TX (US); Linda K. Caldwell, San Antonio, TX (US)

(21) Appl. No.: 13/564,667

(22) Filed: Aug. 1, 2012

Related U.S. Application Data
(60) Provisional application No. 61/515,274, filed on Aug. 4, 2011.

(51) Int. Cl. A61K 9/70

(52) U.S. Cl. CPC A61K 9/70
USPC 424/402; 424/725; 424/729

(57) ABSTRACT

A cooling composition impregnated on a personal towelette which provides a cooling sensation to the body when wiped across the skin. The application of the towelette containing the cooling composition reduces the symptoms of heat due to hot flashes due to menopause, physical exertion, or high temperatures. The composition provides effective cooling but is mild to the skin since it contains active ingredients in combination with skin soothing compounds. The cooling composition of the present invention is made of a combination of natural herbs and botanicals specifically selected to provide synergistic cooling effects.

### CROSS REFERENCE TO RELATED APPLICATIONS

This application claims the benefit under Title 35 United States Code §119(e) of U.S. Provisional Application No. 61/515,274 filed Aug. 4, 2011, the full disclosure of which is incorporated herein by reference.

### BACKGROUND OF THE INVENTION

1. Field of the Invention

The present invention relates generally to cooling compositions used to alleviate heat symptoms. The present invention relates more specifically to a personal towelette impregnated with a cooling composition of herbs and botanicals for use in relief of heat symptoms caused by hot flashes in peri-menopausal women, physical exertion, or high temperatures. The present invention also relates to methods of making and using such wipes.

2. Description of the Related Art

Although the current market includes a variety of wipes, these wipes are intended and manufactured for the purpose of cleaning the hands as a substitute for hand washing and are not intended to serve as a means of cooling the body. Much effort has been directed to different formulations which contain antibacterial and antimicrobial compounds. Many of these compounds traditionally dry and irritate the skin. Thus, these wipes contain a wide variety of germ-killing compounds and are intended and manufactured for the purpose of cleaning the hands and are not intended to serve as a means of relief from heat due to hot flashes, exertion, or hot temperatures.

It would be desirable to have a personal wipe which could provide an easy and effective means of cooling the skin, decreasing the sensation of heat, and which could be re-activated quickly and simply. Additionally, such a towelette should be easy and economical to use, mild to the skin, and utilize a single-use, disposable product. The towelette should have a good shelf life and also be resistant to microbial growth and evaporation of the cooling compound from the product packaging. Existing products have been unable to deliver a towelette which provides all of these benefits.

### SUMMARY OF THE INVENTION

The present invention discloses a cooling composition impregnated on a personal towelette which provides a cooling sensation to the body when wiped across the skin. The application of the towelette containing the cooling composition reduces the symptoms of heat due to hot flashes due to menopause, physical exertion, or high temperatures. The composition provides effective cooling but is mild to the skin since it contains active ingredients in combination with skin soothing compounds.

The cooling composition of the present invention is composed of natural ingredients, including herbs and botanicals with therapeutic or rejuvenating qualities which provide a cooling sensation upon contact with the skin. The cooling agent is compatible with make-up and will not irritate sensitive skin tissues. The sensation of coolness can be re-activated easily to provide renewed relief from heat symptoms by slightly moistening the previously treated area with water. This composition delivers a comfortable cooling sensation which is effective, mild, and residually active. The cooling towelette is pressed onto the face, back of the neck or onto pulse points such as the wrists, and the effect can be refreshed by dampening the skin area later. The towelette has a smooth clean feeling and the composition is not sticky or smelly. The towelette possesses a lengthy shelf life and resists the growth of microbes. The product packaging minimizes the evaporation of the cooling compound from the towelette.

A soft, non-irritating absorbent sheet is impregnated with the cooling composition. The towelette is sized and structured for ease of handling and to adequately cover the surface of an adult user's hand to provide convenience and ease of application. When drawn across the skin, the absorbent towelette draws perspiration away from the skin while dispensing the cooling composition on the skin to provide a cooling effect. The ingredients in the composition are deposited on the skin surface in the process, enabling the user to reactivate the cooling sensation at a later time by simply dampening the area of skin to which the towelette was previously applied. Such a towelette would be easy to transport, dispense, use, and is readily disposable and environmentally friendly. The cooling solution dissipates the heat symptoms associated with hot flashes, physical exertion, or high temperatures and reduces the discomfort caused by such flushing.

### BRIEF DESCRIPTION OF THE DRAWINGS

A more complete understanding of the personal towelette with cooling composition of the present invention may be had by reference to the following detailed description taken in conjunction with the accompanying drawings, wherein:

FIG. 1 is a flow chart disclosing the steps involved in preparation of the active composition of the present invention; and

FIG. 2 is a flow chart disclosing the steps involved in the integration of the final mixture on the personal towelette and into the packaging.

### DETAILED DESCRIPTION OF THE PREFERRED EMBODIMENTS

The present invention discloses a personal towelette containing a cooling composition and a method of making, dispensing, and using the cooling composition. The absorbent sheets used in the present invention are impregnated with the cooling composition. The term “cooling composition” as used herein means a composition suitable for application to the skin for the purpose of cooling the skin surface and decreasing the perception of heat. The cooling composition of the present invention is made of a unique combination of natural herbs and botanicals specifically selected to provide a synergistic cooling effect and satisfy the mildness requirements of the invention. Numerous combinations of active ingredients were tested both in the laboratory and on test participants in order to achieve the maximum synergistic cooling effect. This particular combination of active ingredients is key to the efficacy of the cooling composition, wherein the synergy of the natural herbs and botanicals results in a maximum cooling effect.

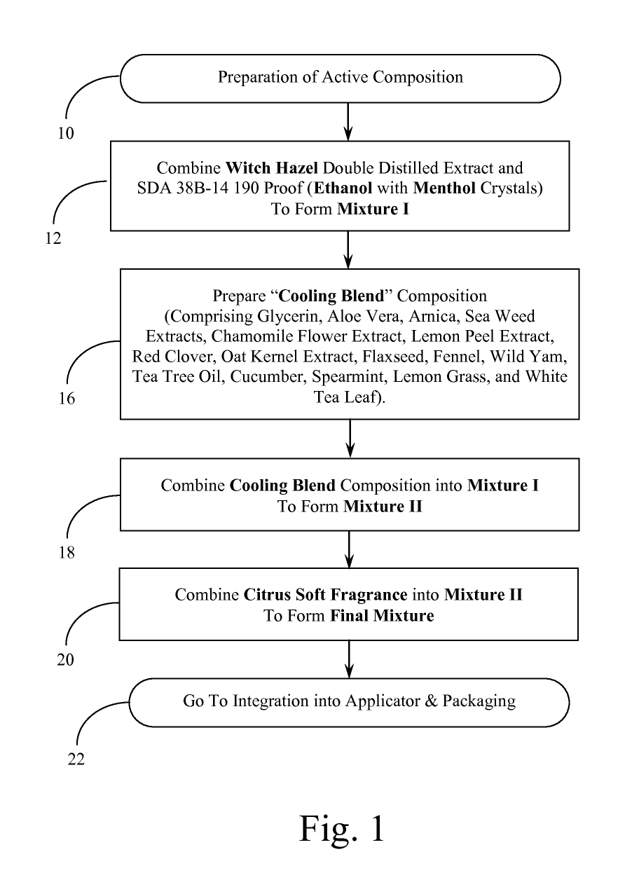

Specifically, as shown in FIG. 1, the cooling composition of a preferred embodiment of the present invention is a mixture comprised of the following components: Witch Hazel Double Distilled Extract, SDA 38B-14, 190 proof (ethanol with menthol crystals), Citrus Soft Fragrance, and a Cooling Blend composition. The preferred embodiment of the proportions of the components is: Witch Hazel Double Distilled Extract 44-60%, SDA 38B-14 36-52%, Citrus Soft Fragrance 0.1-1.0%, and Cooling Blend composition 1-15%. The Cooling Blend composition is comprised of: Glycerin, Aloe Vera, Arnica, Sea Weed Extracts, Chamomile Flower Extract, Lemon Peel Extract, Red Clover, Oat Kernel Extract, Flaxseed, Fennel, Wild Yam, Tea Tree Oil, Cucumber, Spearmint, Lemon Grass, and White Tea Leaf.

The steps for making the active cooling composition 10 begin with combining the Witch Hazel Double Distilled Extract and SDA 38B-14 and mixing 12 until uniform to form Mixture I.

Next, the Cooling Blend composition is prepared 16. As stated above, this composition includes the following: Glycerin, Aloe Vera, Arnica, Sea Weed Extracts, Chamomile Flower Extract, Lemon Peel Extract, Red Clover, Oat Kernel Extract, Flaxseed, Fennel, Wild Yam, Tea Tree Oil, Cucumber, Spearmint, Lemon Grass, and White Tea Leaf. Once this Blend is prepared, it is added 18 to the Mixture I to form Mixture II. Citrus Soft Fragrance is combined 20 into Mixture II to form the Final Mixture. The Final Mixture is blended until all ingredients are dissolved. As previously described, it is the unique combination of the natural herbs and botanicals which provides the synergistic cooling effect of the present invention.

Witch Hazel is an astringent produced from the bark and leaves of the North American Witch Hazel Shrub (Hamamalis Virginiana). The witch hazel leaf and bark are rich in tannins that have both toning and strengthening effects on the skin's proteins and can heal damaged skin. It relieves sluggish circulation and supports the skin's barrier function. Witch Hazel is highly astringent, helping to shrink and contract blood vessels back to normal size. Thus it is known for its soothing, anti-inflammatory properties and can lock in the moisture on the surface of the skin. Witch hazel contains tannins, procyanadins, resin, and flavonoids, all of which add to its soothing, anti-inflammatory properties.

Menthol's ability to chemically trigger the cold-sensitive TRPM8 receptors in the skin is responsible for the well known cooling sensation that it provokes when inhaled, eaten, or applied to the skin. This stimulation of the cold sensors occurs without causing an actual change in temperature. Menthol has analgesic properties that are mediated through a selective activation of κ-opioid receptors. Menthol also enhances the efficacy of ibuprofen in topical applications via vasodilation, which reduces skin barrier function.

Denatured Alcohol serves as a natural emulsifier or binding agent. It has a cooling effect through evaporation and allows the other ingredients in the cooling composition to penetrate the skin.

Glycerin is a building block of all fats and oils. Glycerin acts as a humectant, which is a substance that pulls moisture to the skin.

Aloe Vera is a member of the Lily and Onion family known for use in first aid treatment especially as a topical application. It has been well known for over 4000 years for its therapeutic and healing properties that sooth and softens the skin. Aloe provides a hypoallergenic skin-soothing component for additional mildness to the skin.

Arnica is a botanical in which the roots and flowers are used for a relieving effect. It is calming to muscles, strains or bruises and reduces inflammation.

Sea Weed Extracts is a multi-cellular plant or algae widely known for its healing properties. Seaweed helps ease the pain associated with aches and pains including arthritis and rheumatism. Seaweed is also a known source of iodine, calcium and magnesium.

Chamomile Flower Extract is widely used as a sleep aid and a soother for the stomach. It also has antimicrobial and antioxidant properties and helps to alleviate uncomfortable menstrual cramps.

Lemon Peel Extract is considered an herbal panacea as it is antiseptic, cleansing, toning and strengthening to connective skin tissues. It may firm the skin's elasticity and gives an uplifting fresh fragrance.

Red Clover is an herb considered to be one of the richest sources of isoflavones (water-soluble chemicals that act like estrogens). It is used for lowering cholesterol and improving blood circulation. It helps to mitigate hot flashes and PMS. Also, red clover has been used to treat premature aging of the skin.

Oat Kernel Extract is from a grain which is grown for its seed. When rubbed on the skin, oat extract can be used to remove dead skin cells.

Flaxseed oil is rich in healing and contains high levels of lignans and Omega 3 fatty acids. This benefits the heart; possesses anti-cancer properties; and treats acne, rosacea and eczema. It promotes healthy hair and can help with impotence, infertility and male prostate problems.

Fennel seeds are rich in plant estrogens and have been used to sooth and cool irritated skin. Fennel is aromatic, improves bodily function and was used to boost sexual desire in the past.

Wild Yam is a twining tuberous vine that is used for regulating the liver and endocrine system. It helps reduce the symptoms of menopause and morning sickness, and has long been used to treat infertility.

Tea Tree Oil is an essential oil which is recognized as a potent antiseptic and anti-fungal agent. It is used in shampoos to regulate dandruff. It is also effective in mollifying the skin from minor bee stings, sunburn and irritations.

White Tea Leaf is a natural anti-bacterial plant which is also effective as an energy-booster.

Cucumber is an edible fruit from the gourd family which has many health benefits. It is 90% water and is known for its cooling effects. It is useful in reducing swelling of the eyes and it effective in soothing and softening the skin.

Spearmint is a plant whose scientific name is Mentha Spicata. Essential Oil of Spearmint has been used for centuries for its medicinal properties including antiseptic, anti-spasmodic, disinfectant, relaxation, cooling, and tissue repair.

Lemon Grass is an herb known for its sharp, lemony scent. It is useful for various skin issues and is an effective astringent. It is anti-microbial, anti-bacterial, and anti-fungal.

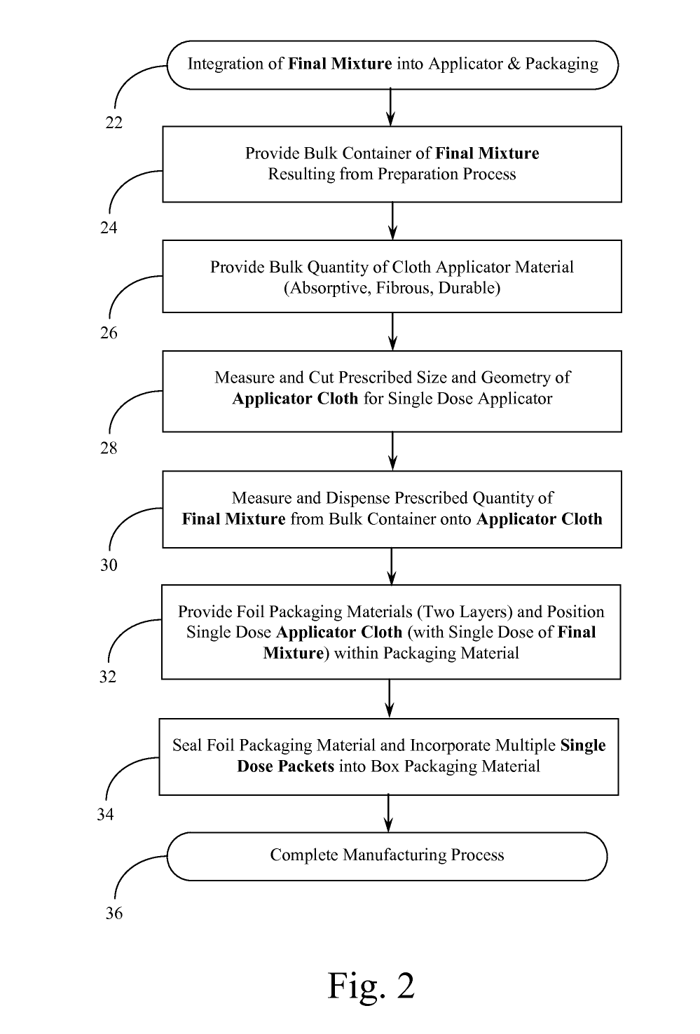

The integration 22 of the Final Mixture into the Applicator and Packaging is shown in FIG. 2. A bulk container of Final Mixture resulting from the preparation process is provided 24. A bulk quantity of cloth applicator material is provided which is absorptive, fibrous, durable, and environmentally friendly 26. The applicator cloth may be formed from any woven or non-woven fiber or fiber mixture of sufficient wet strength and absorbency to hold an effective amount of the composition, about five (5) ml. Additionally, it is important that the sheet possess sufficient tensile strength to reduce the likelihood of tearing the towelette. The manufacture of materials suitable for retention of liquid compounds is well known in the art.

The towelette is made of a wicking material which is capable of removing perspiration and that does not shred, tear, or disintegrate or leave a fibrous residue and which is readily impregnated with the cooling composition. In a preferred embodiment, the towelette is comprised of a white non-apertured spunlace of 65% rayon, 35% polyester, and having a fabric weight of 55.00 (GMS) and which is FDA approved for cosmetics and gentle to sensitive skin. A towelette of the type of material described will be gentle on the skin and at the same time will have a frictional yet non-irritating quality sufficient to stimulate the surface of the skin so as to be more receptive of the cooling composition.

The applicator cloth is measured and cut into the prescribed size and shape for a single dose applicator 28. The sheets are sized and shaped to hold an adequate amount of the cooling composition and provide ease of use for an adult. The towelette is approximately 6.0 inches by 6.0 inches. A measured amount of the Final Mixture, about five (5) ml., is dispensed from the bulk container onto the applicator cloth 30.

Any manufacturing method suitable for the application of aqueous or aqueous/alcoholic impregnates, including flood coating, spray coating, metered dosing, or other techniques known in the art, can be used to impregnate the absorbent sheets of the present invention with the cooling composition. After coating, the sheets are folded and packaged in individual moisture and vapor impermeable packets which are notched or perforated for tearing. The packets are designed to prevent the evaporation of the cooling composition from the personal towelette.

In general, the present invention provides a cooling composition on a personal towelette which may be used in any situation where an individual is overheated. It is anticipated that variations in the cooling composition will be apparent to those skilled in the art from the present disclosure and the attached drawing figures. Such variations, while not explicitly described and defined herein, may be seen to fall within the spirit and scope of the present invention.

We claim:

1. A personal towelette impregnated with a composition of herbs and botanicals for use in relief of heat symptoms caused by hot flashes in peri-menopausal women, physical exertion, or high temperatures, the composition comprising:
an effective amount of witch hazel double distilled extract;
an effective amount of ethanol with menthol crystals;
an effective amount of a Cooling Blend composition of glycerin and botanical extracts and oils; and
an effective amount of a Citrus Soft fragrance.

2. The composition of claim 1 wherein the Cooling Blend composition comprises: Glycerin, Aloe Vera, Arnica, Sea Weed Extracts, Chamomile Flower Extract, Lemon Peel Extract, Red Clover, Oat Kernel Extract, Flaxseed, Fennel, Wild Yam, Tea Tree Oil, Cucumber, Spearmint, Lemon Grass, and White Tea Leaf.

3. The composition of claim 1 wherein the ethanol with menthol crystals is SDA 38B-14, 190 proof.

4. The composition of claim 1 wherein the components are provided in proportions comprising: Witch Hazel Double Distilled Extract 44-60%, SDA 38B-14 36-52%, Citrus Soft Fragrance 0.1-1.0%, and Cooling Blend composition 1-15%.
